(19) United States
(12) Patent Application Publication
(10) Pub. No.: US 2007/0196849 A1
(43) Pub. Date: Aug. 23, 2007

(54) Double-ligation Method for Haplotype and Large-scale Polymorphism Detection

(75) Inventor: Eugene G. Spier, Palo Alto, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/677,987

(22) Filed: Feb. 22, 2007

Related U.S. Application Data
(60) Provisional application No. 60/775,881, filed on Feb. 22, 2006.

(51) Int. Cl. C12Q 1/68

(52) U.S. Cl. 435/6

(57) ABSTRACT

The present teachings provide methods, compositions, and kits for querying the identity of a target polynucleotide strand comprising. In some embodiments, the present teachings provide a method comprising forming a reaction complex comprising the target polynucleotide strand hybridized to an upstream probe, a middle probe, and a downstream probe, wherein the middle probe comprises, A) a first target specific portion, B) a second target specific portion, C) a non-target specific portion, wherein the non-target specific portion is located between the first target specific portion and the second target specific portion, wherein the downstream probe comprises a 5′ end that is adjacent with the 3′ end of the middle probe, wherein the upstream probe comprises a 3′ end that is adjacent with the 5′ end of the middle probe; ligating the upstream probe to the middle probe and the middle probe to the downstream probe to form a ligation product; detecting the ligation product; and, determining the identity of the target polynucleotide strand.

### CROSS REFERENCE TO RELATED APPLICATIONS

This application claims a priority benefit under 35 U.S.C. §119(e) from U.S. Patent Application No. 60/775,881 filed Feb. 22, 2006, which is incorporated herein by reference.

### FIELD

The present teachings relate to methods, compositions, and kits for determining the identity of nucleotides of interest on a target polynucleotide strand.

### INTRODUCTION

Sequencing of the human genome, the HapMap project, and various technical advances allowing whole genome association studies, have lead to an ever expanding appreciation of the number of polymorphisms that are linked to medical conditions and phenotypic traits. Many single nucleotide polymorphisms (SNPs), multiple nucleotide polymorphisms (MNPs), copy number polymorphisms (CNPs), Loss of Heterozygosity (LOH), and large-scale polymorphisms will eventually move to the clinic, and become applicable in medically-relevant applications for patients. Improved approaches for elucidating the identity of polymorphic variations will be imperative to provide improved patient care in the area of clinical diagnostics.

### SUMMARY

A method for determining the identity of a target polynucleotide strand comprising; forming a reaction complex comprising the target polynucleotide strand hybridized to an upstream probe, a middle probe, and a downstream probe, wherein the middle probe comprises, A) a first target specific portion, B) a second target specific portion, C) a non-target specific portion, wherein the non-target specific portion is located between the first target specific portion and the second target specific portion and wherein the non-target specific portion comprises at least five nucleotides, wherein the downstream probe comprises a 5′ end that is adjacent with the 3′ end of the middle probe, wherein the upstream probe comprises a 3′ end that is adjacent with the 5′ end of the middle probe; ligating the upstream probe to the middle probe and the middle probe to the downstream probe to form a ligation product; detecting the ligation product; and, determining the identity of the target polynucleotide strand.

### DRAWINGS

FIGS. 1-4 depict various illustrative embodiments according to some embodiments of the present teachings.

### DESCRIPTION OF EXEMPLARY EMBODIMENTS

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. The term and/or means that the terms before and after can be taken together or separately. For illustration purposes, but not as a limitation, “X and/or Y” can mean “X” or “Y” or “X and Y”.

The section headings used herein are for organizational purposes only and are not to be construed as limiting the described subject matter in any way. All literature and similar materials cited in this application, including, patents, patent applications, articles, books, treatises, and internet web pages are expressly incorporated by reference in their entirety for any purpose. In the event that one or more of the incorporated literature uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Some Definitions

As used herein, the term “nucleotide of interest” refers to nucleotide whose identity is to be determined. For example, the identity of a base at a single nucleotide polymorphism (SNP) locus corresponding to an allele is a nucleotide of interest.

As used herein, the term “discriminating nucleotide” refers to a nucleotide contained in the target specific portion of a probe that can query a nucleotide of interest by base-pairing with that nucleotide of interest.

As used herein, the term “middle probe” refers to a probe that queries the target polynucleotide strand by hybridization, and which contains a first target specific portion, a second target specific portion, and a non-target specific portion located between the first target specific portion and the second target specific portion. In some embodiments, the middle probe can be a connector allele-specific oligonucleotide probe (connector ASO probe), and can contain, for example, a discriminating nucleotide in its 3′ end. In some embodiments, the discriminating nucleotide, when present, can reside at the terminus of the 3′ end. Optionally, additional zipcode and/or primer portion sequence information can be included in a middle probe.

As used herein, the term “upstream probe” refers to a probe that queries the target polynucleotide strand by hybridization, and which contains a target specific portion, and optionally additional zipcode and/or primer portion sequence information. In some embodiments, the upstream probe can be an allele-specific oligonucleotide probe (ASO probe), and can contain, for example, a discriminating nucleotide at its 3′ end. In some embodiments, the discriminating nucleotide, when present, can reside at the terminus of the 3′ end.

As used herein, the term “downstream probe” refers to a probe that queries the target polynucleotide strand by hybridization, and which contains a target specific portion, and optionally additional zipcode and/or primer portion sequence information. In some embodiments, the downstream probe can be a locus-specific oligonucleotide probe (LSO probe), and can contain, for example, a discriminating nucleotide at its 5′ end. In some embodiments, the discriminating nucleotide, when present, can reside at the terminus of the 5′ end.

As used herein, the term “non-target specific portion of the middle probe” refers to a sequence of nucleotides that is between the first target specific portion and the second target specific portion of the middle probe, and which is not complementary to the target polynucleotide strand.

In some embodiments, the present teachings provide a method for determining the identity of a target polynucleotide strand comprising; forming a reaction complex comprising the target polynucleotide strand hybridized to an upstream probe, a middle probe, and a downstream probe, wherein the middle probe comprises, A) a first target specific portion, B) a non-target specific portion, wherein the non-target specific portion is at least five nucleotides in length and wherein the non-target specific portion is located between the first target specific portion and the second target specific portion, C) a second target specific portion; wherein the downstream probe comprises a 5′ end that is adjacent with the 3′ end of the middle probe, wherein the upstream probe comprises a 3′ end that is adjacent with the 5′ end of the middle probe; ligating the upstream probe to the middle probe and the middle probe to the downstream probe to form a ligation product; detecting the ligation product; and, determining the identity of the target polynucleotide strand.

In some embodiments, the present teachings provide an approach for determining the identity of two distantly located nucleotides of interest on a target polynucleotide strand. A ligation reaction can be performed wherein three oligonucleotide probes are hybridized to a target polynucleotide strand. The middle probe can comprise a first target specific portion at its 5′ end, and a second target specific portion at its 3′ end. The middle probe can further comprise a non-target specific portion located between the first target specific portion and the second target specific portion. This non-target specific portion can comprise a zip-code, thus facilitating decoding of the resulting ligation product and determination of the nucleotides of interest. The middle probe allows for the bringing together of a first region of the target polynucleotide strand containing the first nucleotide of interest, with a second region of the target polynucleotide strand containing the second nucleotide of interest. A stretch of sequence of the target polynucleotide strand, referred to as a “non-hybridized loop region,” is located between these two nucleotides of interest, and does not hybridize to any of the three probes. On either side of the middle probe is an upstream probe and a downstream probe. When the upstream probe hybridizes to the target polynucleotide strand, and the downstream probe hybridizes to the target polynucleotide strand, and the middle probe hybridizes to the target polynucleotide strand, a complex suitable for ligation can form. Ligating the three probes forms a ligation product, the detection of which allows for the determination of the two nucleotides of interest. By having discriminating nucleotides located in at least two of the ligation probes in such a position to effect ligation, the generation of a ligation product can be indicative of the presence of particular nucleotides of interest.

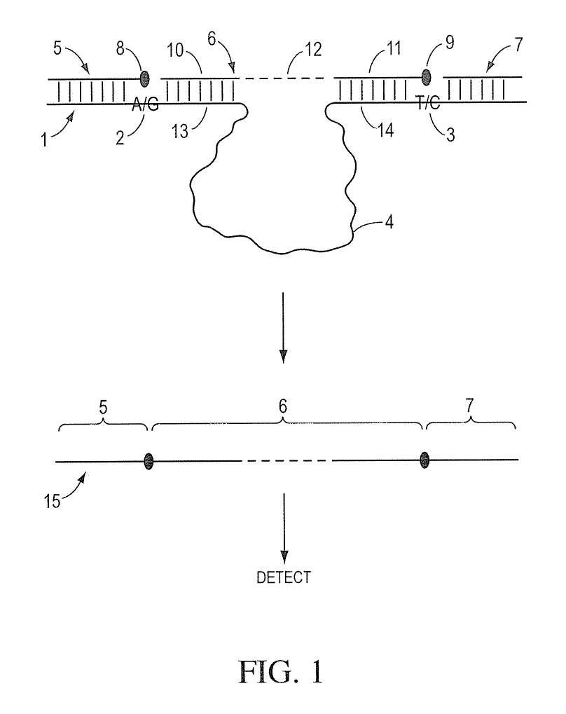

An illustrative embodiment of this approach is shown in FIG. 1. Here, a target polynucleotide strand (1) contains a first nucleotide of interest (2, an A or a G), and a second nucleotide of interest (3, a T or a C). The two nucleotides of interest can be considered single nucleotide polymorphisms (SNPs). These SNPs are separated on their strand by several nucleotides by a non-hybridized loop region (4). The target polynucleotide strand (1) is shown hybridized to three probes, an upstream probe, here termed an allele-specific oligonucleotide probe (ASO probe (5)), a middle probe, here termed a connector ASO probe (6), and a downstream probe, here termed a locus specific oligonucleotide probe (LSO probe (7)). The ASO probe (5) contains a discriminating nucleotide at its 3′ terminus (filled circle, 8), which in this case would be either a T or a C since the corresponding nucleotide of interest (2) is an A or a G. The connector ASO (6) contains a discriminating nucleotide at its 3′ terminus (filled circle, 9), which in this case would be either an A or a G since the corresponding nucleotide of interest (3) is a T or a C. The connector ASO probe contains a first target specific portion (10), a second target specific portion (11), and a non-target specific portion (12, dashed). The non-target specific portion of the connector ASO (12) does not generally hybridize to the target polynucleotide strand. Thus the first target specific portion of the connector ASO (10) and the second target specific portion of the connector ASO (11) can hybridize to two different non-continuous regions of the target polynucleotide strand (13 and 14), thus bringing the two SNPs within query-able range with the three ligation probes. Accordingly, ligation of the ASO probe (5) to the connector ASO probe (6), and the connector ASO probe (6) to the LSO probe (7), can occur in a situation where the target polynucleotide strand comprises an A at the first SNP and a T at the second SNP, and correspondingly the ASO probe contains a T discriminating nucleotide and the connector ASO probe contains an A discriminating nucleotide. The result of these two ligation events is a ligation product (15). The ligation product contains the ASO probe (5), the connector ASO probe (6), and the LSO probe (7). Detection of this ligation product provides for the identification of the first SNP and the second SNP in the target polynucleotide strand.

Subsequent to the ligation reaction, any number of procedures can be employed to removed unligated probes. For example, in some embodiments the 5′ end of the ASOs and the 3′ end of the LSO can be protected to confer nuclease resistance. As a result, unligated connector ASO's, unligated ASOs, and unligated LSOs can be susceptible to various 5′ and/or 3′ acting nucleases. Also subsequent to ligation, any number of amplification procedures can be employed to produce additional copies of the ligation product, for example PCR. Thus, it will be appreciated that that when the present teachings refer to detecting the ligation product, such detection can generally involve the ligation products, as well as surrogates thereof, including amplification products such as PCR amplicons.

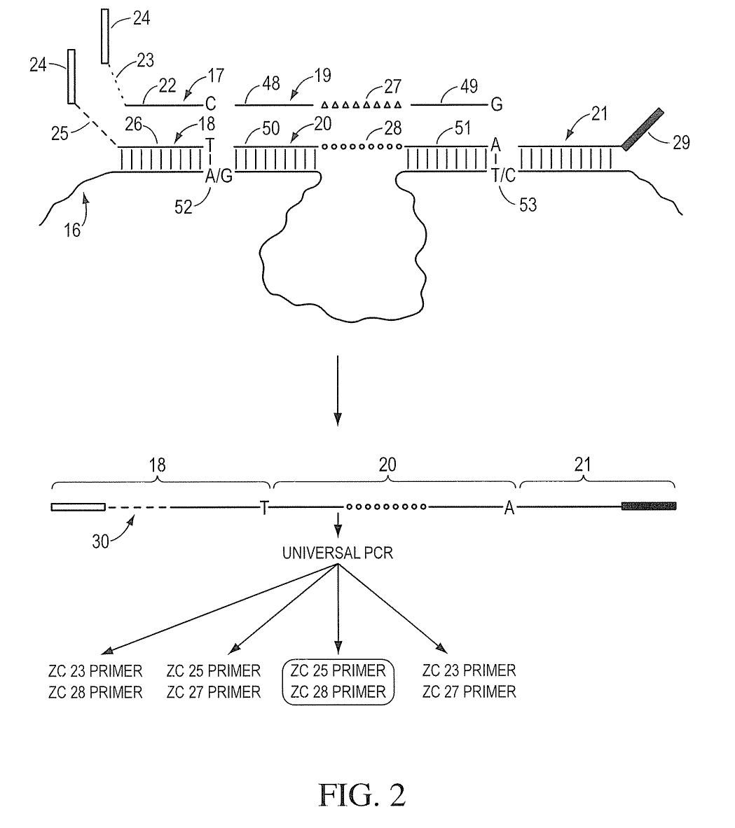

Another illustrative embodiment is shown in FIG. 2. Here, the identity of a first SNP (52, A or G) and a second SNP (53, T or C) on a target polynucleotide strand (16) is queried. The ligation probes employed include a first ASO probe (17), a second ASO probe (18), a first connector ASO probe (19), a second connector ASO probe (20), and an LSO probe (21). The depicted reaction architecture comprises two ligation events on the target polynucleotide strand. Depending on the identity of the nucleotides of interest at the first SNP (52) and the second SNP (53) on the target polynucleotide strand, a given ASO probe and a given connector ASO probe will hybridize and become ligated together, as well as ligated to the downstream LSO. Because the ASO probes, connector ASO probes, and LSO probe can comprise distinct zip-codes, the identity of the resulting ligation product can be ascertained through a decoding reaction that employs zipcode reagents. Thus, the first ASO probe (17) contains a target specific portion (22) that hybridizes to the target polynucleotide strand, a discriminating nucleotide (C), a first ASO zipcode (23, dotted), and a universal forward primer portion (24, open rectangle). The second ASO (18) contains a target specific portion (26) that hybridizes to the target polynucleotide strand, a discriminating nucleotide (T), a second ASO zipcode (25, dashed), and a universal forward primer portion (24, open rectangle). If an A is present as the nucleotide of interest at the first SNP (52), then the second ASO probe (18) will hybridize to the target polynucleotide strand and be suitable for ligation to a connector ASO probe. The decision of which connector ASO probe the ASO probe ligates to is determined by the nature of the nucleotide of interest at the second SNP (53), and correspondingly whether the first connector ASO probe (19) or the second connector ASO probe (20) contains the appropriate discriminating nucleotide to hybridize to the nucleotide of interest at that second SNP. Here, the first connector ASO probe (19) contains a first connector ASO zipcode (27, triangles) between the first target specific portion (48) and the second target specific portion (49). The first connector ASO probe (19) further contains a G as its discriminating nucleotide. The second connector ASO probe (20) contains a second connector ASO zipcode (28, circles) between the first target specific portion (50) and the second target specific portion (51). The second connector ASO probe (20) further contains an A as its discriminating nucleotide. The LSO probe (21) will hybridize to the target polynucleotide strand, and become ligated to the corresponding connector ASO. The LSO probe contains a universal reverse primer portion (29, darkened rectangle). Thus, ligation produces a ligation product which contains the zipcode of the incorporated ASO probe, and also contains the zipcode of the incorporated connector ASO probe. A decoding reaction can thus determine the identity of the two SNPs based on these zipcodes. Any number of various decoding reactions can be performed. For example, when a plurality of different target polynucleotide strands are queried in a multiplex ligation reaction to produce a collection of different zipcoded ligation products, it can be desirable to perform a universal PCR, using for example a universal forward primer (encoded by 24 bof the ASO probes) and a universal reverse primer (encoded by 29 of the LSO probe). Thereafter, a collection of lower plex decoding PCRs can be performed in separate wells in a reaction plate, where each well contains a particular configuration of zipcode primers. The well containing an amplicon in this decoding PCR will identity the two SNPs in the target polynucleotide strand. For example, as shown, four decoding PCRs can be performed.

The first decoding PCR can contain a zipcode primer 23 (ZC 23) and a zipcode primer 28 (ZC 28). A product resulting from this first decoding PCR would indicate the presence of a G allele at the first SNP and a T allele at the second SNP.

The second decoding PCR can contain a zipcode primer 25 (ZC 25) and a zipcode primer 27 (ZC 27). A product resulting from this second decoding PCR would indicate the presence of an A allele at the first SNP and a C allele at the second SNP.

The third decoding PCR can contain a zipcode primer 25 (ZC 25) and a zipcode primer 28 (ZC 28). A product resulting from this third decoding PCR would indicate the presence of an A allele at the first SNP and a T allele at the second SNP. This third decoding PCR is shown circled, indicating the presence of a PCR product (for example by the presence of signal from an interchelating dye such as Sybr Green), thus reflecting amplification of the ligation product (30), itself containing the complementary T and A nucleotides, respectively.

The fourth decoding PCR can contain a zipcode primer 23 (ZC 23) and a zipcode primer 27 (ZC 27). A product resulting from this fourth decoding PCR would indicate the presence of a G allele at the first SNP and a C allele at the second SNP.

Illustrative teachings of such zipcode-based PCR decoding approaches can be found in U.S. patent application Ser. No. 11/090,468 to Lao, and U.S. patent application Ser. No. 11/090,830 to Andersen. Descriptions of zip-codes can be found in, among other places, U.S. Pat. Nos. 6,309,829 (referred to as “tag segment” therein); 6,451,525 (referred to as “tag segment” therein); 6,309,829 (referred to as “tag segment” therein); 5,981,176 (referred to as “grid oligonucleotides” therein); 5,935,793 (referred to as “identifier tags” therein); and PCT Publication No. WO 01/92579 (referred to as “addressable support-specific sequences” therein).

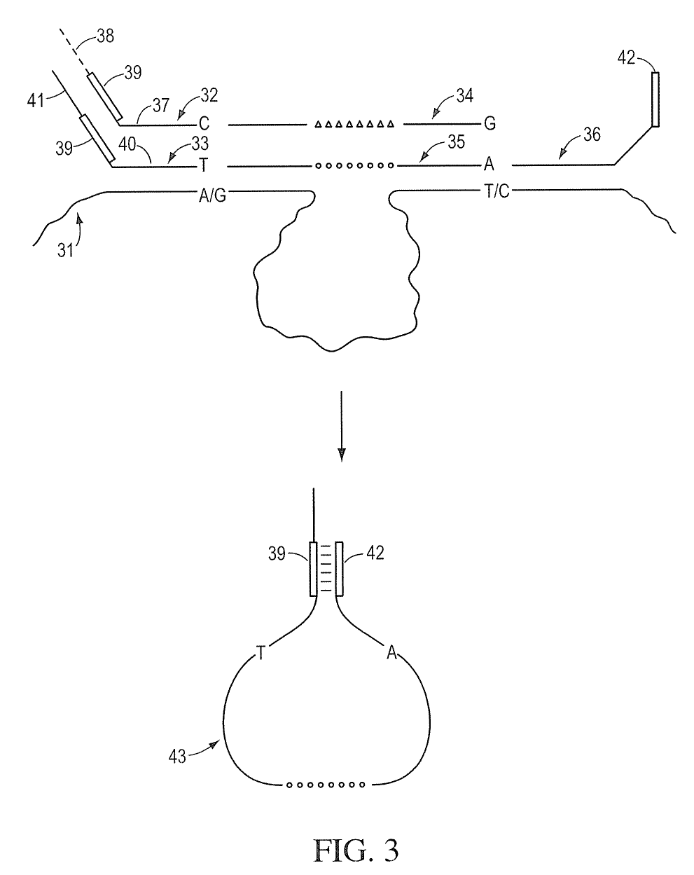

Another embodiment of the present teachings is provided in FIG. 3. Here, the reaction results in a ligation product that comprises a self-complementary region. In FIG. 3, the identity of a first SNP (A or G) and a second SNP (T or C) on a target polynucleotide strand (31) is queried. The ligation probes employed include a first ASO probe (32), a second ASO probe (33), a first connector ASO probe (34), a second connector ASO probe (35), and an LSO probe (36). The first ASO probe (32) contains a first target specific portion (37) that can hybridize to the target polynucleotide strand, a discriminating nucleotide (C), a first ASO zipcode (38, dashed), and a nascent self-complementary portion (39, open rectangle). The second ASO probe (33) contains a target specific portion (40) that hybridizes to the target polynucleotide strand, a discriminating nucleotide (T), a second ASO zipcode (41, line), and a nascent self-complementary portion (39, open rectangle). The first connector ASO probe (34) and second connector ASO probe (35) can comprise elements as discussed earlier. Finally, the LSO probe (36) can hybridize to the target polynucleotide strand, and become ligated to the corresponding connector ASO probe. The LSO probe contains a nascent self-complementary portion (42, open rectangle). Thus, a ligation product results which can form a self-complementary structure (43), where the nascent self-complementary portion of the incorporated ASO probe (39) and the nascent self-complementary portion of the LSO probe (42) hybridize. A decoding reaction can thus determine the identity of the SNPs, for example decoding based on these zipcodes. Any number of various decoding reactions can be performed. Additional illustrative teachings for making and detecting self-complementary ligation products can be found in Spier, U.S. Pat. No. 7,169,561.

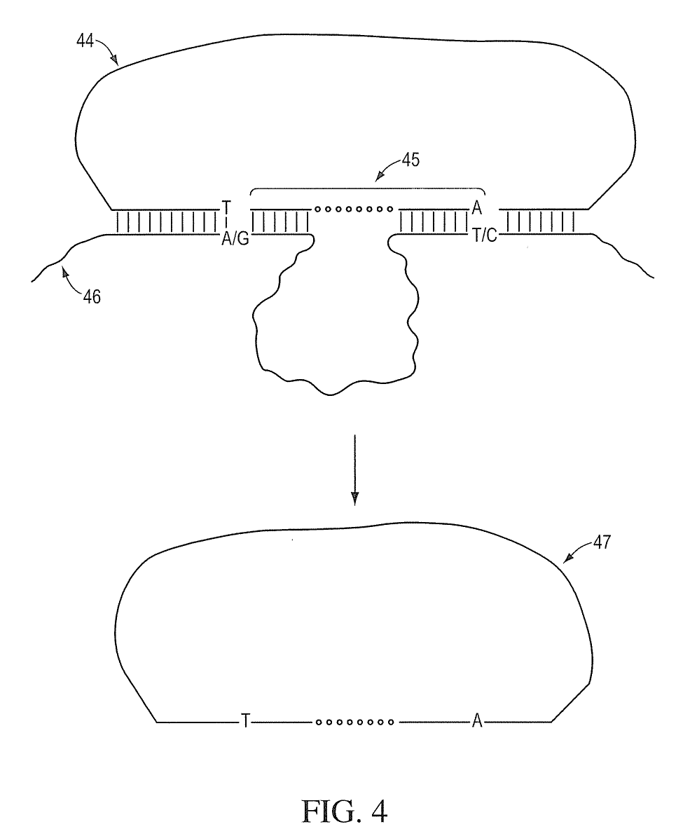

Another embodiment according to the present teachings is shown in FIG. 4. Here, the ASO probe can be connected to the LSO probe as a single molecule, a combined ASO-LSO probe (44). Thus, hybridization of the connector ASO probe (45) to the target polynucleotide strand, along with the combined ASO-LSO probe (44), results in a substrate suitable for ligation. The two ligation sites, sealed by ligase, provide for the generation of a circular ligation product (47). Thus, in some embodiments the ligation probes of the present teachings can be designed such that a ligation product forms a circularized molecule. The resulting circularized ligation products can contain any of a number of zipcode strategies for decoding, as described elsewhere herein. The circular ligation products can also be amplified, for example by rolling circle amplification, prior to detection. Illustrative rolling circle amplification methods can be found for example in U.S. Pat. No. 5,854,033 and U.S. Pat. No. 6,797,474. Approaches for forming circular ligation products in the context of conventional OLA-approaches can be found in U.S. Pat. No. 5,871,921 to Landegren, where the ligation probes are referred to as padlock probes.

Additionally, the present teachings can also be employed to query the identity of nucleotides of interest in a target polynucleotide strand between two different samples, as is further described in U.S. patent application Ser. No. 11/090,468 to Lao, and U.S. patent application Ser. No. 11/090,830 to Andersen. For example, zipcodes can be used not only to encode nucleotides of interest in the target polynucleotide strand, but can also be used to encode the identity of the sample from which the target polynucleotide strand is derived. Thus, a normal sample can be directly compared to a disease sample for example. As another example, a first patient's DNA can be encoded with a first zipcode and a second patient's DNA can be encoded with a second zipcode.

It will be appreciated that the decoding reaction can comprise any of a number of methods known in the field of molecular biology, and in general the nature of such decoding is not a limitation of the present teachings. One example of a decoding scheme that can be employed in the context of the present teachings employs PCR. Here, a PCR amplification of the ligation product can be performed using a biotinylated primer. The resulting amplicon thus can comprise two strands, one of which is biotinylated. The amplicon can be immobilized, for example on a streptavidin-containing solid support. The non-biotinylated strand of the amplicon can then be removed. Hybridization of a “zipchute” molecule comprising a sequence complementary to a zipcode present on one of the ligation probes can then be performed. Such a zipchute can further comprise a distinct mobility modifier, and label (such as a florophore). Washing of unhybridized zipchutes, and subsequent elution of the bound zipchute, can then be followed by analysis of the eluted zipchute by a mobility dependant analysis technique such as capillary electrophoresis, thereby allowing for the identification of the nucleotides of interest in the target polynucleotide strand. Illustrative teachings of such zipchute-based decoding approaches can be found in U.S. patent application Ser. No. 09/584,905 to Wenz, and U.S. Pat. Nos. 6,759,202 and 6,756,204 to Grossman.

Another example of a decoding scheme that can be employed in the context of the present teachings employs simply measuring the ligation product in a mobility dependant analysis technique. For example, the length of the ligation probes can be varied according to the identity of the nucleotides of interest, such that the size of the product encodes the identity of the nucleotides of interest. Additional description of such approaches employing such “stuffer” sequences in ligation probes can be found in Schouten, U.S. Pat. No. 6,955,901.

In some embodiments, the zip-coded ligation products can be amplified in a PCR, wherein a label is included on one of the PCR primers. The resulting labeled amplicons can then be detected on a solid support such as a zipcode array. Illustrative ligation approaches with zipcode array read-out is discussed in U.S. Pat. No. 6,852,487 to Barany. In some embodiments, array-based readouts can be performed where each element (spot) on the array comprises an oligonucleotide that contains two zipcodes.

In some embodiments, the ligation products can be “pre-amplified” in a multiplexed PCR. Examples of pre-amplification can be found in WO2004/051218 to Andersen and Ruff, U.S. Pat. No. 6,605,451 to Gerdes. In some embodiments, the products of such a pre-amplification reaction can be decoded with secondary single-plex decoding PCRs.

In some embodiments, there are five or more nucleotides on the target polynucleotide strand between the first nucleotide of interest and the second nucleotide of interest. In some embodiments, there are ten or more nucleotides on the target polynucleotide strand between the first nucleotide of interest and the second nucleotide of interest. In some embodiments, there are fifteen or more nucleotides on the target polynucleotide strand between the first nucleotide of interest and the second nucleotide of interest. In some embodiments, there are twenty or more nucleotides on the target polynucleotide strand between the first nucleotide of interest and the second nucleotide of interest. In some embodiments, there are thirty or more nucleotides on the target polynucleotide strand between the first nucleotide of interest and the second nucleotide of interest. In some embodiments, there are fifty or more nucleotides on the target polynucleotide strand between the first nucleotide of interest and the second nucleotide of interest. In some embodiments, there are one hundred or more nucleotides on the target polynucleotide strand between the first nucleotide of interest and the second nucleotide of interest. In some embodiments, there are two hundred or more, three hundred or more, four hundred or more, or five hundred or more nucleotides on the target polynucleotide strand between the first nucleotide of interest and the second nucleotide of interest.

In some embodiments, the non-target specific portion of the connector ASO probe contains at least five nucleotides. In some embodiments, the non-target specific portion of the connector ASO probe contains at least ten nucleotides. In some embodiments, the non-target specific portion of the connector ASO probe contains at least twelve nucleotides. In some embodiments, non-target specific portion of the connector ASO probe contains at least fifteen nucleotides. In some embodiments, the non-target specific portion of the connector ASO probe contains at least twenty nucleotides. In some embodiments, the non-target specific portion of the connector ASO probe contains at least thirty nucleotides, at least fifty nucleotides, at least one hundred nucleotides, at least two hundred nucleotides, or more.

The present teachings also contemplate embodiments in which greater than two nucleotides of interest are identified on a single target polynucleotide strand by the formation of a single ligation product. For example, in some embodiments three nucleotides of interest are queried and identified for a single target polynucleotide strand. For example, in some embodiments four nucleotides of interest are queried and identified for a single target polynucleotide strand. For example, in some embodiments five nucleotides of interest are queried and identified for a single target polynucleotide strand. For example, in some embodiments greater than five nucleotides of interest are queried and identified for a single target polynucleotide strand. Increasing the number of nucleotides queried on a single target polynucleotide strand can be achieved, for example, by increasing complexity in the zip-code based encoding scheme and various decoding approaches, along with increasing the number of ASO probes and connector ASO probes.

The present teachings also contemplate embodiments in which two or more nucleotides of interest are identified on a first single target polynucleotide strand, and two or more nucleotides of interest are identified on a second single target polynucleotide strand. Such approaches involved multiplexed ligation reactions in which a collection of different single target polynucleotide strands are queried to form a collection of different ligation products. Encoding the ligation products with zipcodes and/or primer portions allows for their decoding, and correspondingly the elucidation of the identity of a large number of nucleotides of interest, for example occurring at a large number of different SNP loci on a large number of different target polynucleotide strands.

In some embodiments, for example when two or more SNPs are the nucleotide of interest, it will be appreciated that greater than two nucleotides are possible for a given SNP. Indeed, up to four nucleotides can be present at a given SNP, the A, G, C, or T that comprise DNA. Thus, the present teachings can provide, for example, a collection of four different ASO probes to query the four different nucleotides that could exist at a given SNP locus.

In some embodiments, the relationship between the ASO and connector ASO, and/or the relationship between the connector ASO and the LSO, when hybridized to the target polynucleotide strand, is such that a nucleotide overlap (a “flap”) exists. Flap endonucleases (Fen) can thus be employed to removed overlapping nucleotides, thus creating a suitable substrate for ligation. Illustrative teaching describing the use of flap endonucleases with ligation can be found in U.S. Pat. No. 6,511,810.

In general, it will be appreciated that the location of the discriminating nucleotide in the ligation probes is free to vary according to routine modifications in experimental design. For example, in some embodiments, the discriminating nucleotide is located at the 3′ terminus of an ASO probe. That is, the 3′ terminus of a first ASO probe can contain a first discriminating nucleotide for a first SNP, and the 3′ terminus of a second ASO probe can contain a second discriminating nucleotide for that first SNP. However, the discriminating nucleotide need not be at the 3′ terminus of the ASO probe. In some embodiments, the discriminating nucleotide can reside in the interior of the probe. In some embodiments, the downstream probe (an LSO probe) can contain a discriminating nucleotide at the terminus that is adjacent to the connector ASO. In some embodiments, the downstream probe can comprise a discriminating nucleotide in its interior. The presence of a discriminating nucleotide in the interior of a probe, as opposed to a probes' terminus, can allow for hybridization stringency to provide an additional level of selectivity in the ligation reaction. Varying the location of the discriminating nucleotide in the connector ASO probe is also contemplated by the present teachings.

In some embodiments, a polymerase can be included in the ligation reaction, for example a non strand-displacing polymerase. Thus, the ligation probes can be designed such that gaps exist between the hybridized probes. Filling in of these gaps by the polymerase can allow for the probes to become suitable for ligation. Illustrative teachings of “gap-ligation” can be found in U.S. Pat. No. 5,427,930. Thus, as used in the present teachings, probes that are said to be hybridized “adjacent” to one another can broadly refer to situations in which the probes are directly adjacent (contiguous), as well as situations in which the hybridized probes have small gaps of a 1, or 2, or 3, or 4, or 5 or greater nucleotides.

In some embodiments where a circular ligation product is formed, for example is depicted in FIG. 4, amplification and detection of the circular ligation product can proceed by a concatenation procedure as discussed in U.S. Patent Application 2004/0029142A1 to Schon.

The target polynucleotide strands of the present teachings can come from any of a variety of sample materials. Genomic DNA and RNA from any organism, or non-living substance, can be employed. Many methods are available for the isolation and purification of target polynucleotide strands for use in the present invention. Preferably, the target polynucleotide strands are sufficiently free of proteins and any other interfering substances to allow adequate target-specific primer annealing and extension. Exemplary purification methods include (i) organic extraction followed by ethanol precipitation, e.g., using a phenol/chloroform organic reagent (Ausubel), preferably with an automated DNA extractor, e.g., a Model 341 DNA Extractor available from PE Applied Biosystems (Foster City, Calif.); (ii) solid phase adsorption methods (Walsh, 1991; Boom); and (iii) salt-induced DNA precipitation methods (Miller), such methods being typically referred to as “salting-out” methods. Optimally, each of the above purification methods is preceded by an enzyme digestion step to help eliminate protein from the sample, e.g., digestion with proteinase K, or other proteases. Other desirable methods of purification include use of NucPrep™ Chemistry from Applied Biosystems, through the ABI Prism™ 6100 Nucleic Acid PrepStation or the ABI Prism™ 6700 Automated Nucleic Acid Workstation.

Further, the present teachings contemplate embodiments in which prior to ligation the target polynucleotide strand is treated with bisulfite, and the first nucleotide of interest, the second nucleotide of interest, or both the first nucleotide of interest and the second nucleotide of interest are converted from an unmethylated cytosine to a uracil. Illustrative methods of performing methylation analysis on bisulfite-treated samples can be found in published US Patent Application US20050095623A1, published US Patent Application US20050079527A1, and US Patent Application US20060121492A1.

The present teachings can also be employed to query particular splice variants. For example, the first target specific portion of the connector ASO probe can correspond to a first exon, the second target specific portion of the connector ASO probe can correspond to a second exon, and the non-target specific portion of the connector ASO probe can correspond to an intron. Illustrative methods of designing probes for delineating genomic DNA, introns, exons, and splice variants generally can be found in U.S. Pat. No. 6,258,543 and U.S. Pat. No. 6,063,568.

The present teachings can be applied to determining polymorphisms in any of a variety of forms, including single nucleotide polymorphisms (SNPs), multiple nucleotide polymorphisms (MNPs), copy number polymorphisms (CNPs), Loss of Heterozygosity (LOH), and large-scale polymorphisms.

Generally, illustrative ligation and amplification approaches can be found in published US Patent Application US20050064459A1 and U.S. Pat. No. 6,696,470.

### Certain Exemplary Kits

The instant teachings also provide kits designed to expedite performing certain of the disclosed methods. Kits may serve to expedite the performance of certain disclosed methods by assembling two or more components required for carrying out the methods. In certain embodiments, kits contain components in pre-measured unit amounts to minimize the need for measurements by end-users. In some embodiments, kits include instructions for performing one or more of the disclosed methods. Preferably, the kit components are optimized to operate in conjunction with one another.

For example in some embodiments the present teachings comprises a kit comprising; a middle probe, an upstream probe, and a downstream probe. In some embodiments, the kit further comprises a ligase. In some embodiments, the kit further comprises reagents for performing a PCR, said reagents comprising primers, nucleotides, polymerase, and buffer.

Although the disclosed teachings have been described with reference to various applications, methods, and kits, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

We claim:

1. A method for determining the identity of a target polynucleotide strand comprising;
forming a reaction complex comprising the target polynucleotide strand hybridized to an upstream probe, a middle probe, and a downstream probe,
wherein the middle probe comprises,
A) a first target specific portion,
B) a second target specific portion,
C) a non-target specific portion, wherein the non-target specific portion is located between the first target specific portion and the second target specific portion and wherein the non-target specific portion comprises at least five nucleotides,
wherein the downstream probe comprises a 5′ end that is adjacent with the 3′ end of the middle probe,
wherein the upstream probe comprises a 3′ end that is adjacent with the 5′ end of the middle probe;
ligating the upstream probe to the middle probe and the middle probe to the downstream probe to form a ligation product;
detecting the ligation product; and,
determining the identity of the target polynucleotide strand.

2. The method according to claim 1 wherein;
the 3′ end of the upstream probe comprises a first discriminating nucleotide that base-pairs with a first nucleotide of interest;
wherein the 3′ end of the middle probe comprises a second discriminating nucleotide that base-pairs with a second nucleotide of interest; or,
both the 3′ end of the upstream probe comprises a first discriminating nucleotide that base-pairs with the first nucleotide of interest and the 3′ end of the middle probe comprises a second discriminating nucleotide that base-pairs with the second nucleotide of interest.

3. The method according to claim 2 wherein;
the 3′ end of the upstream probe comprises the first discriminating nucleotide at its terminus;
the 3′ end of the middle probe comprises the second discriminating nucleotide at its terminus; or
both the first discriminating nucleotide is located at the terminus of the 3′ end of the upstream probe and the second discriminating nucleotide is located at the terminus of the 3′ end of the middle probe.

4. The method according to claim 1 wherein the upstream probe comprises a 5′ primer portion, and the downstream probe comprises a 3′ primer portion, and wherein the ligation product is amplified in a PCR with a primer pair corresponding with the 5′ primer portion of the upstream probe and the 3′ primer portion of the downstream probe.

5. The method according to claim 2 wherein prior to ligation the target polynucleotide strand is treated with bisulfite, and the first nucleotide of interest, the second nucleotide of interest, or both the first nucleotide of interest and the second nucleotide of interest are converted from an unmethylated cytosine to a uracil.

6. The method according to claim 2 wherein the first nucleotide of interest and the second nucleotide of interest are a first SNP locus and a second SNP locus, and the determining comprises identifying a haplotype.

7. A method for determining the identity of a first nucleotide of interest and a second nucleotide of interest in a target polynucleotide strand comprising;
forming a reaction complex comprising the target polynucleotide strand hybridized to an upstream probe, a middle probe, and a downstream probe,
wherein the middle probe comprises,
A) a first target specific portion,
B) a second target specific portion, wherein the second target specific portion comprises a 3′ end, wherein the 3′ end comprises a discriminating nucleotide that base-pairs with the first nucleotide of interest,
C) a non-target specific portion, wherein the non-target specific portion is located between the first target specific portion and the second target specific portion and wherein the non-target specific portion comprises at least five nucleotides,
wherein the downstream probe comprises a 5′ end that is adjacent with the 3′ end of the middle probe,
wherein the upstream probe comprises a 3′ end that is adjacent with the 5′ end of the middle probe, and wherein the 3′ end comprises a discriminating nucleotide that base-pairs with the second nucleotide of interest;
ligating the upstream probe to the middle probe and the middle probe to the downstream probe to form a ligation product;
detecting the ligation product; and,
determining the identity of the first nucleotide of interest and the second nucleotide of interest in the target polynucleotide strand.

8. The method according to claim 7 wherein;
the 3′ end of the upstream probe comprises the first discriminating nucleotide at its terminus;
the 3′ end of the middle probe comprises the second discriminating nucleotide at its terminus; or
both the first discriminating nucleotide is located at the terminus of the 3′ end of the upstream probe and the second discriminating nucleotide is located at the terminus of the 3′ end of the middle probe.

9. The method according to claim 7 wherein the upstream probe comprises a 5′ primer portion, the downstream probe comprises a 3′ primer portion, and wherein the ligation product is amplified in a PCR with a primer pair corresponding with the 5′ primer portion of the upstream probe and the 3′ primer portion of the downstream probe.

10. The method according to claim 7 wherein prior to ligation the target polynucleotide strand is treated with bisulfite, and the first nucleotide of interest, the second nucleotide of interest, or both the first nucleotide of interest and the second nucleotide of interest are converted from an unmethylated cytosine to a uracil.

11. The method according to claim 7 wherein the first nucleotide of interest and the second nucleotide of interest are a first SNP locus and a second SNP locus, and the determining comprises identifying a haplotype.

12. A middle probe comprising;
a first target specific region, a non-target specific region, and a second target specific region, wherein the non-target specific region is at least five nucleotides in length and is located between the first target specific region and the second target specific region, and wherein at least one of the first target specific portion and the second target specific portion further comprises a discriminating nucleotide.

13. The middle probe according to claim 12 wherein the discriminating nucleotide is at the 3′ end of the middle probe.

14. The middle probe according to claim 13 wherein the discriminating nucleotide is at the terminus of the 3′ end of the middle probe.

15. A composition comprising a first middle probe and a second middle probe, the composition comprising;
the first middle probe, wherein the first middle probe comprises a first target specific portion, a non-target specific portion, and a second target specific portion, wherein the non-target specific portion is at least five nucleotides in length and is located between the first target specific portion and the second target specific portion, and wherein at least one of the first target specific portion of the first middle probe and the second target specific portion of the first middle probe further comprises a first discriminating nucleotide; and,
the second middle probe, wherein the second middle probe comprises the first target specific portion, a second non-target specific portion, and the second target specific portion, wherein the second non-target specific region is at least five nucleotides in length and is located between the first target specific portion and the second target specific portion, and wherein at least one of the first target specific portion and the second target specific portion further comprises a second discriminating nucleotide,
wherein the first target specific portion of the first middle probe is the same sequence as the first target specific portion of the second middle probe;
wherein the second target specific portion of the first middle probe is the same sequence as the second target specific portion of the second middle probe;
wherein the non-target specific portion of the first middle probe is a different sequence from the non-target specific portion of the second middle probe; and,
wherein the first discriminating nucleotide of the first middle probe is different from the second discriminating nucleotide of the second middle probe.

16. The composition according to claim 15 wherein the first discriminating nucleotide is at the 3′ terminus of the first middle probe, wherein the second discriminating nucleotide is at the 3′ terminus of the second middle probe, or both the first discriminating nucleotide is at the 3′ terminus of the first middle probe and the second discriminating nucleotide is at the 3′ terminus of the second middle probe

17. A kit for identifying a first nucleotide of interest and a second nucleotide of interest on a single target polynucleotide strand, the kit comprising;
an upstream probe, a middle probe, and a downstream probe,
wherein the middle probe comprises a first target specific portion, a non-target specific portion, and a second target specific portion, wherein the non-target specific portion is at least five nucleotides in length and is located between the first target specific portion and the second target specific portion, and wherein at least one of the first target specific portion of the middle probe and the second target specific portion of the middle probe further comprises a first discriminating nucleotide.

18. The kit according to claim 17 further comprising a ligase.

19. The kit according to claim 17 further comprising reagents for a PCR, said reagents comprising a primer pair, nucleotides, polymerase, and buffer.
